(19) United States
(12) Patent Application Publication
(10) Pub. No.: US 2022/0167946 A1
(43) Pub. Date: Jun. 2, 2022

(54) ULTRASONIC IMAGE DISPLAY SYSTEM AND PROGRAM

(71) Applicant: GE Precision Healthcare LLC, Wauwatosa, WI (US)

(72) Inventors: Shunichiro Tanigawa, Tokyo (JP); Naohisa Kamiyama, Tokyo (JP)

(21) Appl. No.: 17/529,034

(22) Filed: Nov. 17, 2021

(30) Foreign Application Priority Data
Nov. 27, 2020 (JP) 2020-197245

(51) Int. Cl. A61B 8/08; A61B 8/00

(52) U.S. Cl. CPC A61B 8/5246; A61B 8/488; A61B 8/463; A61B 8/5207

(57) ABSTRACT

A processor in an ultrasonic image display system displays in first and second B-mode images BI1, BI2 a first measured portion M1 and a region R indicating a candidate of a second measured portion M2, and after the first measured portion M1 has been displayed, acquires a result of a first measurement based on echo data obtained by a first measuring scan. Moreover, after the region R has been displayed, the processor stores echo data obtained by a second measuring scan in said memory. After the echo data obtained by the second measuring scan has been stored, once a user interface has accepted an operator's input for defining the second measured portion M2 within the region R, the processor acquires a result of a second measurement based on the echo data stored in the memory.

### FIELD OF THE INVENTION

The present invention relates to an ultrasonic image display system for acquiring a plurality of measurement values, and a program for controlling the same.

### BACKGROUND OF THE INVENTION

In examinations using an ultrasonic diagnostic apparatus, which is an example of an ultrasonic image display system, hardness of biological tissue, for example, is measured based on echo signals from ultrasound to perform a diagnosis on a liver parenchymal portion, or the like.

Moreover, in examinations using the ultrasonic diagnostic apparatus, analysis is sometimes performed by acquiring a plurality of measurement values, rather than only one measurement value. For example, for evaluation of the liver parenchymal portion, at least two of measurement values including hardness, viscosity, attenuation, a speckle pattern, a brightness level, and a subcutaneous thickness are acquired at the same time for one cross section, and a comprehensive diagnosis is performed using them.

In acquiring the measurement values, an operator sometimes defines a measured portion, which serves as an object of a measurement, in an ultrasonic image. In this case, the operator experiences stress in defining a portion for every one of the plurality of measurements that is suitable for the measurement.

### BRIEF SUMMARY OF THE INVENTION

According to an aspect of the invention, an ultrasonic image display system includes a processor, an ultrasonic probe, a display, memory, and a user interface, wherein said processor is adapted to control said ultrasonic probe to perform a B-mode scan for a B-mode image on a patient, and to display on said display at least one B-mode image based on echo data obtained by said B-mode scan. The processor is also adapted to display in said B-mode image a first measured portion serving as an object of a first measurement and a region indicating a candidate of a second measured portion serving as an object of a second measurement. Moreover, the processor is adapted to: after said first measured portion has been displayed, control said ultrasonic probe to perform a first measuring scan for said first measurement on said patient; acquire a result of the first measurement for said first measured portion based on echo data obtained by said first measuring scan; after said region has been displayed, control said ultrasonic probe to perform a second measuring scan for said second measurement on said patient; and store echo data obtained by said second measuring scan in said memory. Said user interface is adapted to, after the echo data obtained by said second measuring scan has been stored in said memory, accept an operator's input for defining said second measured portion within said region. Said processor is further adapted to, once said user interface has accepted said operator's input, acquire a result of the second measurement for said second measured portion based on the echo data that has been obtained by said second measuring scan and stored in said memory.

According to an aspect, a program for controlling an ultrasonic display system including a processor, an ultrasonic probe, a display, memory, and a user interface is disclosed. The program for controlling is adapted to cause the processor to control said ultrasonic probe to perform a B-mode scan for a B-mode image on a patient. The program for controlling is adapted to cause the processor to display on said display at least one B-mode image based on echo data obtained by said B-mode scan. The program for controlling is adapted to cause the processor to display in said B-mode image a first measured portion serving as an object of a first measurement and a region indicating a candidate of a second measured portion serving as an object of a second measurement. The program for controlling is adapted to cause the processor to, after said first measured portion has been displayed, control said ultrasonic probe to perform a first measuring scan for said first measurement on said patient, and acquiring a result of the first measurement for said first measured portion based on echo data obtained by said first measuring scan. The program for controlling is adapted to cause the processor to, after said region has been displayed, control said ultrasonic probe to perform a second measuring scan for said second measurement on said patient, and store echo data obtained by said second measuring scan in said memory. Said user interface is adapted to, after the echo data obtained by said second measuring scan has been stored in said memory, accept an operator's input for defining said second measured portion in said region. The program for controlling is adapted to cause the processor to perform the act of, once said user interface has accepted said operator's input, acquiring a result of the second measurement for said second measured portion based on the echo data that has been obtained by said second measuring scan and stored in said memory.

### BRIEF DESCRIPTION OF THE DRAWINGS

FIG. 1 is a block diagram showing an example of an ultrasonic image display system according to an embodiment;

FIG. 2 is a flow chart showing an example of processing including acquisition of a result of a first measurement in the ultrasonic image display system according to the embodiment;

FIG. 3 is a diagram showing a display on which a first B-mode image and a second B-mode image are displayed according to an embodiment;

FIG. 4 is a diagram showing the display on which a first measured portion and a region are displayed in the first and second B-mode images according to an embodiment;

FIG. 5 is a diagram showing the display on which an elasticity image is displayed in the first measured portion according to an embodiment;

FIG. 6 is a flow chart showing an example of processing including acquisition of a result of a second measurement in the ultrasonic image display system according to the embodiment;

FIG. 7 is a diagram showing the display on which a second measured portion is defined according to an embodiment;

FIG. 8 is a diagram showing the display on which a B-mode image is displayed according to an embodiment;

FIG. 9 is a diagram showing the display on which a first measured portion and a region are displayed in the B-mode image according to an embodiment; and

FIG. 10 is a diagram showing the display on which a second measured portion is defined according to an embodiment.

### DETAILED DESCRIPTION OF THE INVENTION

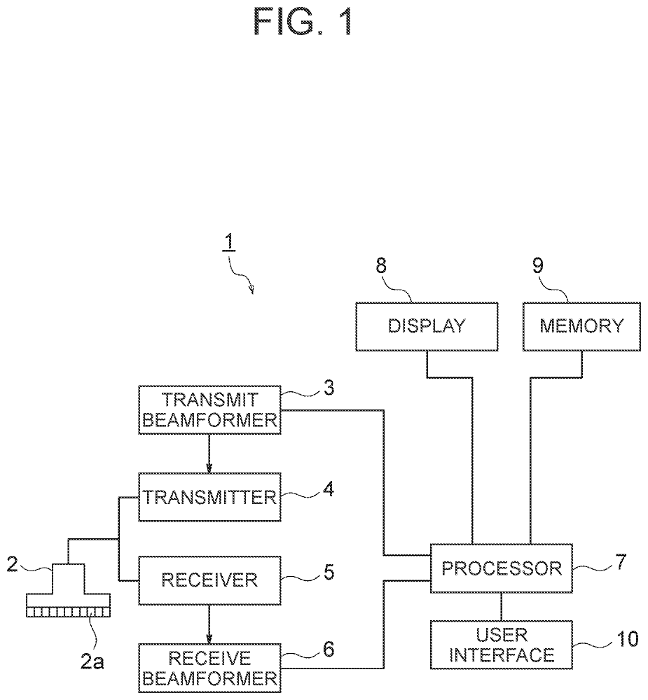

Now embodiments of the present invention will be described hereinbelow with reference to the accompanying drawings. An ultrasonic image display system 1 shown in FIG. 1 is an ultrasonic diagnostic apparatus in an example, and comprises an ultrasonic probe 2, a transmit beamformer 3, and a transmitter 4. The ultrasonic probe 2 performs an ultrasonic scan on a patient, and receives ultrasonic echoes. The ultrasonic scan includes a B-mode scan, a first measuring scan, and a second measuring scan, which will be discussed later.

More specifically, the ultrasonic probe 2 has a plurality of vibration elements 2a for emitting pulsed ultrasound to the patient (not shown). The plurality of vibration elements 2a are driven by the transmit beamformer 3 and transmitter 4 to emit pulsed ultrasound.

The ultrasonic image display system 1 further comprises a receiver 5 and a receive beamformer 6. The pulsed ultrasound emitted from the vibration elements 2a is reflected in the inside of the patient to generate echoes returning to the vibration elements 2a. The echoes are converted into electrical signals by the vibration elements 2a, which are echo signals, and are input to the receiver 5. The echo signals undergo amplification, etc. with a required gain at the receiver 5, and then input to the receive beamformer 6, where receive beamforming is performed. The receive beamformer 6 outputs receive-beamformed ultrasound data.

The receive beamformer 6 may be a hardware beamformer or a software beamformer. In the case that the receive beamformer 6 is a software beamformer, it may comprise one or more processors including one or more of a graphics processing unit (GPU), a microprocessor, a central processing unit (CPU), a digital signal processor (DSP), or any other type of processors capable of executing logical operations. The processor(s) constituting the receive beamformer 6 may be constructed from a processor separate from a processor 7 described later, or constructed from the processor 7.

The ultrasonic probe 2 may comprise electrical circuitry to perform all or part of transmit and/or receive beamforming. For example, all or part of the transmit beamformer 3, transmitter 4, receiver 5, and receive beamformer 6 may be situated within the ultrasonic probe 2.

The ultrasonic image display system 1 also comprises the processor 7 for controlling the transmit beamformer 3, transmitter 4, receiver 5, and receive beamformer 6. Moreover, the ultrasonic image display system 1 comprises a display 8, memory 9, and a user interface 10.

The processor 7 comprises one or more processors. The processor 7 is in electronic communication with the ultrasonic probe 2. The processor 7 may control the ultrasonic probe 2 to acquire ultrasound data. The processor 7 controls which of the vibration elements 2a are active, and the shape of an ultrasonic beam transmitted from the ultrasonic probe 2. The processor 7 is also in electronic communication with the display 8, and the processor 7 may process the ultrasound data into ultrasonic images for display on the display 8. The term “electronic communication” may be defined to include both wired and wireless connections. The processor 7 may include a central processing unit (CPU) according to one embodiment. According to other embodiments, the processor 7 may include other electronic components capable of carrying out processing functions, such as a digital signal processor, a field-programmable gate array (FPGA), a graphics processing unit (GPU), or any other type of processor. According to other embodiments, the processor 7 may include a plurality of electronic components capable of carrying out processing functions. For example, the processor 7 may include two or more electronic components selected from a list of electronic components including: a central processing unit, a digital signal processor, a field-programmable gate array, and a graphics processing unit.

The processor 7 may also include a complex demodulator (not shown) that demodulates RF data. In another embodiment, the demodulation can be carried out earlier in the processing chain.

The processor 7 is adapted to perform one or more processing operations according to a plurality of selectable ultrasonic modalities on the data. The data may be processed in real-time during a scanning session as the echo signals are received. For the purpose of this disclosure, the term “real-time” is defined to include a procedure that is performed without any intentional delay.

The data may be temporarily stored in a buffer (not shown) during ultrasonic scanning, so that they can be processed in a live operation or in an off-line operation not in real-time. In this disclosure, the term “data” may be used in the present disclosure to refer to one or more datasets acquired using an ultrasonic image display system.

The ultrasound data may be processed by the processor 7 in other or different mode-related modules (e.g., B-mode, color Doppler, M-mode, color M-mode, spectral Doppler, contrast-enhanced mode, elastography, TVI, strain, strain rate, and the like) to form data for ultrasonic images. For example, one or more modules may produce ultrasonic images in B-mode, color Doppler, M-mode, color M-mode, spectral Doppler, contrast-enhanced mode, elastography, TVI, strain, strain rate, and combinations thereof, and the like.

The image beams and/or image frames are stored and timing information indicating a time at which the data was acquired in memory may be recorded. The modules may include, for example, a scan conversion module to perform scan conversion operations to convert the image frames from coordinate beam space to display space coordinates. A video processor module may be provided that reads the image frames from memory and displays the image frames in real-time while a procedure is being carried out on the patient. The video processor module may store the image frames in image memory, from which the ultrasonic images are read and displayed on the display 8.

As used herein, the term “image” broadly refers to both of a visible image, and data representing a visible image. The term “data” may include both of raw data that is ultrasound data before the scan conversion operations, and image data that is data after the scan conversion operations.

In the case that the processor 7 includes a plurality of processors, the aforementioned processing tasks to be handled by the processor 7 may be handled by the plurality of processors. For example, a first processor may be utilized to demodulate and decimate the RF signal while a second processor may be used to further process the data prior to displaying an image.

In the case that the receive beamformer 6 is a software beamformer, for example, its processing functions may be carried out by a single processor or by a plurality of processors.

The display 8 is an LED (Light Emitting Diode) display, an LCD (Liquid Crystal Display), an organic EL (Electro-Luminescence) display, or the like.

The memory 9 is any known data storage medium. In an example, the ultrasonic image display system 1 comprises non-transitory storage media and transitory storage media as the memory 9, and comprises a plurality of units of memory 9. The non-transitory storage medium is, for example, a non-volatile storage medium such as an HDD (Hard Disk Drive) and ROM (Read Only Memory). The non-transitory storage media may include a portable storage medium such as a CD (Compact Disk) and a DVD (Digital Versatile Disk). In the non-transitory storage medium, programs executed by the processor 7 are stored.

The transitory storage medium is a volatile storage medium such as RAM (Random Access Memory).

The user interface 10 can accept an operator's input. For example, the user interface 10 accepts an input of a command and/or information from the operator. The user interface 10 is constructed to include a keyboard, hard keys, a trackball, a rotary control, soft keys, and the like. The user interface 10 may include a touch screen that displays soft keys, etc.

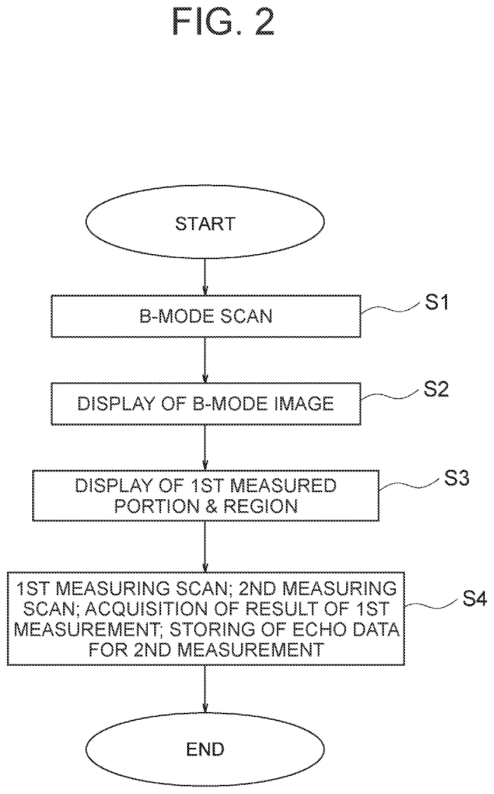

Next, an operation in the ultrasonic image display system 1 in the present embodiment will be described. At Step S1 in the flow chart in FIG. 2, the processor 7 controls the ultrasonic probe 2 to perform a B-mode scan for a B-mode image on a patient. Next, at Step S2, the processor 7 displays a first B-mode image BI1 and a second B-mode image BI2 side by side on the display 8, as shown in FIG. 3, based on echo data obtained by the B-mode scan. In an example, the first and second B-mode images BI1, BI2 are images of the same cross section in the patient and in the same frame. In this case, a B-mode scan for one frame is performed on a certain cross section in the patient at Step S1. However, the first and second B-mode images BI1, BI2 are not limited to the images of the same cross section and in the same frame. For example, the first and second B-mode images BI1, BI2 may be images of the same cross section and in different frames. In this case, a B-mode scan for two frames is performed on a certain cross section in the patient at Step S1.

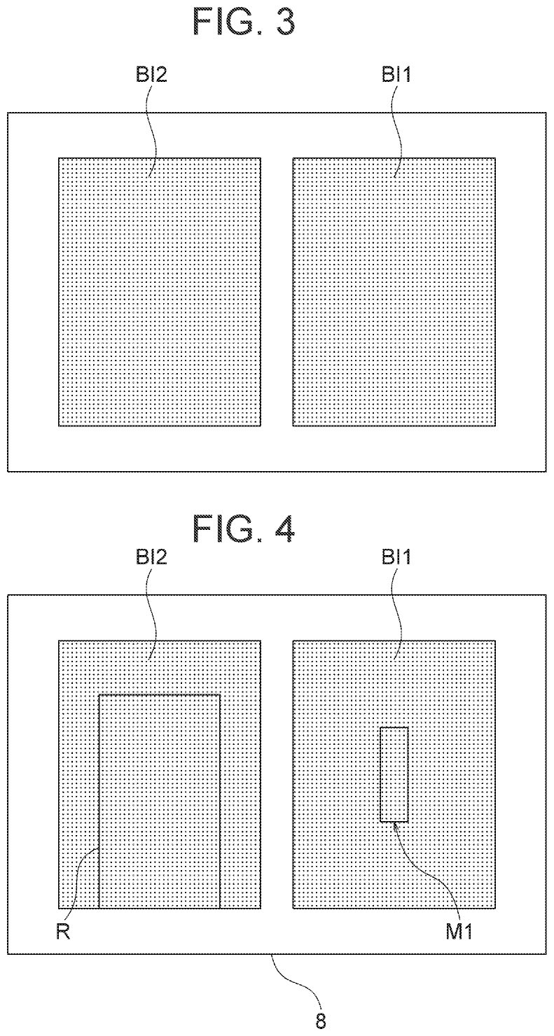

Next, at Step S3, the processor 7 displays a first measured portion M1 in the first B-mode image BI1, and a region R in the second B-mode image BI2, as shown in FIG. 4. The first measured portion M1 is a region serving as an object of a first measurement. Although the first measured portion M1 is a rectangular region in FIG. 4, it is not limited thereto. The region R is a region indicating a candidate of a second measured portion serving as an object of a second measurement. Although the region R is a rectangular region in the drawing, it is not limited thereto. While the first measured portion M1 is an object of the first measurement as a whole, the region R serves as an object of the second measurement not as a whole but a second measured portion that is part thereof serves as the object, which will be discussed later.

Now display and definition of the first measured portion M1 and region R will be described. In an example, once the user interface 10 has accepted an operator's input, the processor 7 displays the first measured portion M1 at a required position in the first B-mode image BI1, and displays the region R at a required position in the second B-mode image BI2. The user interface 10 may be configured to accept an operator's input for fixing the position of the first measured portion M1. In this case, the operator's input may include an input for moving the first measured portion M1 in the first B-mode image BI1. The processor 7 fixes the position of the first measured portion M1 in the first B-mode image BI1 based on the input at the user interface 10. The operator can thus move the first measured portion M1 to a desired position suitable for the first measurement, and define it.

On the other hand, the processor 7 displays the region R at a predetermined position in the second B-mode image BI2. The predetermined position is a position suitable for the second measurement.

Next, at Step S4, the processor 7 controls the ultrasonic probe 2 to perform on the patient a first measuring scan for the first measurement and a second measuring scan for the second measurement. The processor 7 acquires a result of the first measurement for the first measured portion M1 based on echo data obtained by the first measuring scan.

The first measuring scan is performed so as to include at least a region in the patient that corresponds to the first measured portion M1. The second measuring scan is performed so as to include at least a region in the patient that corresponds to the region R.

The first measuring scan is a scan for measuring a value relating to elasticity of the patient's biological tissue, in an example. The second measuring scan is a scan for measuring a value of ultrasound attenuation in the patient's biological tissue, in an example.

The value relating to elasticity of biological tissue is a velocity of propagation of shear waves caused in the biological tissue by an ultrasonic pulse (push pulse) transmitted to the biological tissue, in an example. The first measuring scan includes transmission of the push pulse for generating shear waves in the biological tissue, and transmission/reception of ultrasonic detecting pulses for detecting the shear waves generated by the push pulse in the biological tissue. The velocity of propagation of the shear waves propagating through the biological tissue is calculated based on echo data obtained by transmission of the ultrasonic detecting pulses. The velocity of propagation is an example of the result of the first measurement.

The processor 7 may calculate an elasticity value (Young's modulus (in Pa; Pascal)) of the biological tissue based on the velocity of propagation of the shear waves. The elasticity value is another example of the result of the first measurement.

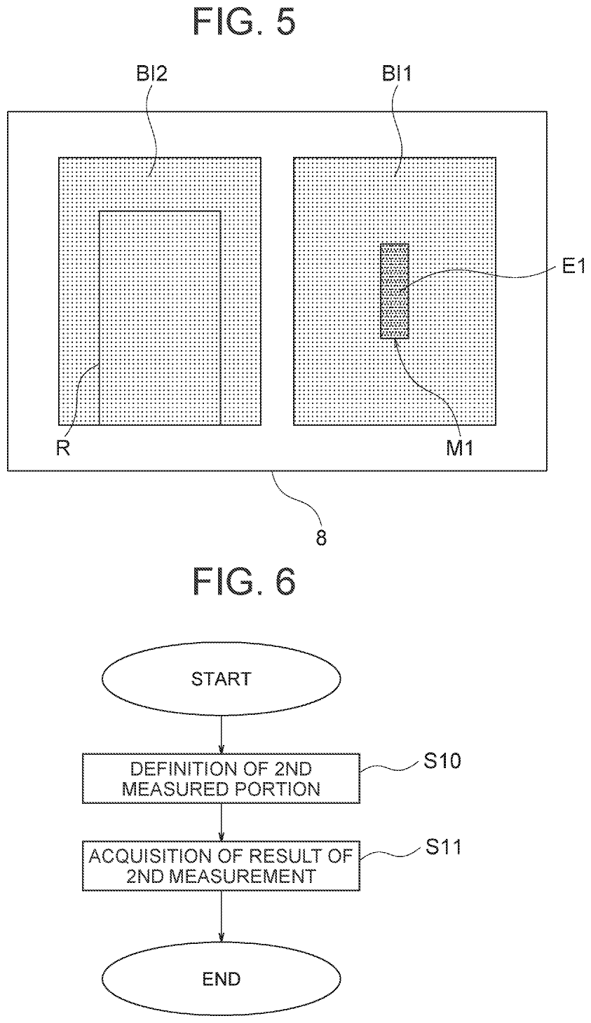

The processor 7 may acquire the result of the first measurement, such as the velocity of propagation or elasticity value, for each of pixels in the region of the first measured portion M1. The processor 7 may produce an elasticity image for the region of the first measured portion M1 based on the velocity of propagation or elasticity value. The processor 7 displays an elasticity image EI in the first measured portion M1 defined in the first B-mode image BI1, as shown in FIG. 5. The elasticity image EI is a semitransparent color image through which the first B-mode image BI1 in the background passes. The color image is an image having colors depending upon the velocity of propagation or elasticity value, which image has colors depending upon elasticity of the biological tissue.

Moreover, at Step S4, the processor 7 further stores the echo data for the second measurement obtained by the second measuring scan in the memory 9. The second measurement is not performed here, and only storage of the echo data for the second measurement is performed. At this time point, the operator does not have to define a second measured portion serving as an object on which a second measurement is to be performed. The only thing that the operator performs for the second measurement by this time point is to confirm whether the second B-mode image BI2 in the region R is suitable for the second measurement i.e., measurement of attenuation. Therefore, the operator can concentrate on the first measurement.

The processor 7 may store in the memory 9 the echo data obtained by the first measuring scan and the result of the first measurement based on the echo data.

Additionally, at Step S4, before the first measuring scan is performed, the processor 7 may control the ultrasonic probe to perform a B-mode scan for a B-mode image in one frame and refresh the first B-mode image BI1 based on echo data based on the B-mode scan. The processor 7 displays the first measured portion M1 in the refreshed first B-mode image BI1 as well.

Moreover, at Step S4, before the second measuring scan is performed, the processor 7 may control the ultrasonic probe to perform a B-mode scan for a B-mode image in one frame and refresh the second B-mode image BI2 based on echo data based on the B-mode scan. The processor 7 displays the region R in the refreshed second B-mode image BI2 as well.

For example, at Step S4, scans may be performed in the order of: a B-mode scan for one frame, a first measuring scan for one frame, a B-mode scan for one frame, and a second measuring scan for one frame. Moreover, scans for a plurality of frames may be performed by a sequence comprising: a B-mode scan for one frame, a first measuring scan for one frame, a B-mode scan for one frame, and a second measuring scan for one frame, the sequence being repeated in this order.

The first measurement is thus completed by Steps S1 to S4 above. After completing the first measurement, a result of the second measurement is acquired following the flow chart in FIG. 6. In an example, the processing following the flow chart in FIG. 6 is started while displaying the first and second ultrasonic images BI1, BI2, and elasticity image EI, as shown in FIG. 5.

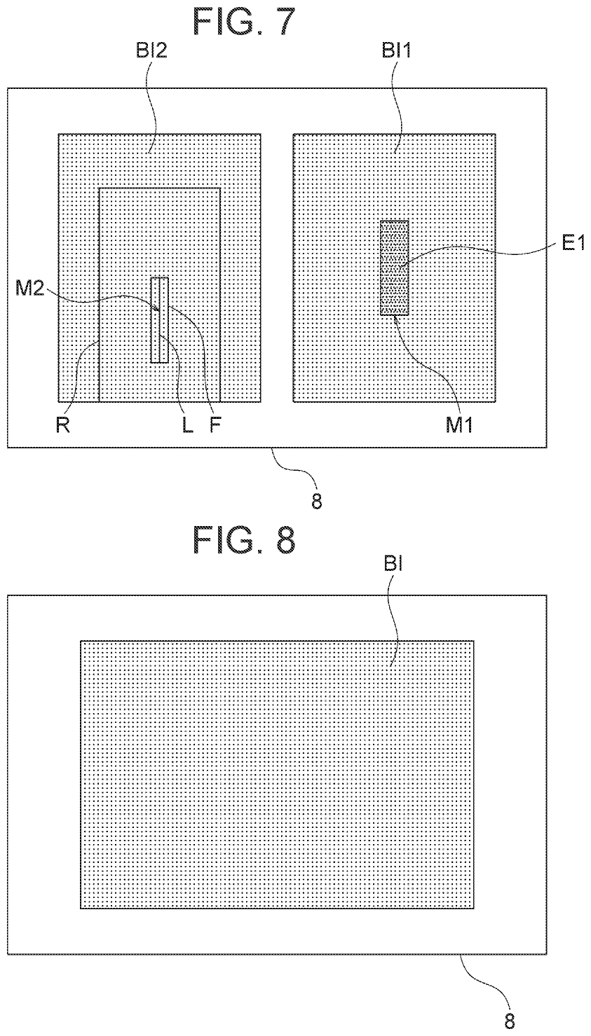

At Step S10, a second measured portion M2 serving as an object of the second measurement is defined. Once the user interface 10 has accepted an operator's input, the processor 7 defines the second measured portion M2 in the second B-mode image BI2, as shown in FIG. 7. The second measured portion M2 is defined at a desired position in the region R, which is constituted by a line segment L in a box F. The line segment L extends in a direction of an acoustic line for ultrasound transmitted/received at the ultrasonic probe 2.

Next, at Step S11, the processor 7 acquires a result of the second measurement for the second measured portion M2 based on the echo data that has been obtained by the second measuring scan and stored in the memory 9. The result of the second measurement is a value of ultrasound attenuation, which is the degree of attenuation of echo signals along the portion of the line segment L. The resulting attenuation value may be displayed on the display 8.

As above, the operator only has to define the second measured portion M2 at Step S10 after the second measuring scan has been completed and the echo data has been stored in the memory 9 at Step S4. Thus, stress of the operator can be reduced.

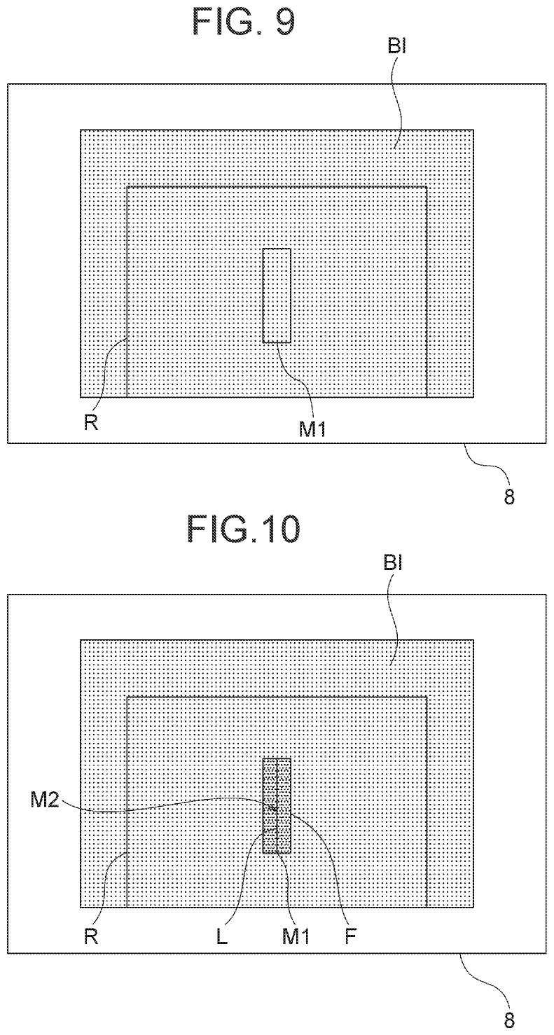

Next, a variation will be described. At Step S2 described earlier, the processor 7 may display on the display 8 only one B-mode image BI, as shown in FIG. 8, in place of the first B-mode image BI1 and second B-mode image BI2 displayed side by side. In this case, at Step S3, the processor 7 displays the first measured portion M1 and region R in the B-mode image BI, as shown in FIG. 9. Moreover, at Step S10, the second measured portion M2 is defined in the region R displayed in the B-mode image BI, as shown in FIG. 10. In FIG. 10, the second measured portion M2 is defined within the region of the first measured portion M1. However, the position at which the second measured portion M2 is defined is not limited to the inside of the region of the first measured portion Ml. Furthermore, the position at which the first measured portion M1 is defined is not limited to the inside of the region R.

According to various embodiments, the first measurement may be a measurement of a value of ultrasound attenuation in biological tissue, and the second measurement may be a measurement of an elasticity-related value in the biological tissue. Moreover, the first and second measurements are not limited to the measurements of the attenuation- or elasticity-related value. For example, the first and second measurements may be echo data-based measurements that are any combination of measurements of different kinds including: measurements relating to elasticity, viscosity, a subcutaneous thickness, and ultrasound attenuation in patient's biological tissue; a measurement of asperities of a patient's liver surface; and analysis of texture of the aforesaid B-mode image. Analysis of texture of the B-mode image includes measurements of a speckle pattern and brightness in the B-mode image.

While the present invention has been described with reference to particular embodiments, various changes may be made and/or equivalents may be substituted without departing from the scope and spirit of the present invention. In addition, many modifications may be made to adapt a particular situation or material to the teachings of the invention, without departing from the scope and spirit of the present invention. Therefore, the present invention is not limited to the particular embodiments disclosed herein, and it is intended that the present invention will encompass all the embodiments falling within the appended claims.

1. An ultrasonic image display system comprising a processor, an ultrasonic probe, a display, memory, and a user interface, wherein
said processor is adapted to perform control comprising the acts of:
controlling said ultrasonic probe to perform a B-mode scan for a B-mode image on a patient;
displaying on said display at least one B-mode image based on echo data obtained by said B-mode scan;
displaying in said B-mode image a first measured portion serving as an object of a first measurement and a region indicating a candidate of a second measured portion serving as an object of a second measurement;
after said first measured portion has been displayed, controlling said ultrasonic probe to perform a first measuring scan for said first measurement on said patient, and acquiring a result of the first measurement for said first measured portion based on echo data obtained by said first measuring scan; and
after said region has been displayed, controlling said ultrasonic probe to perform a second measuring scan for said second measurement on said patient, and storing echo data obtained by said second measuring scan in said memory,
said user interface is adapted to, after the echo data obtained by said second measuring scan has been stored in said memory, accept an operator's input for defining said second measured portion in said region, and
said processor is adapted to, once said user interface has accepted said operator's input, acquire a result of the second measurement for said second measured portion based on the echo data that has been obtained by said second measuring scan and stored in said memory.

2. The ultrasonic image display system as recited in claim 1, wherein:
said user interface is adapted to further accept an operator's input for fixing a position of said first measured portion displayed in said B-mode image, and said input includes an operator's input for moving said first measured portion on said B-mode image, and
said processor
fixes the position of said first measured portion in said B-mode image based on said input, and
displays said region at a predetermined position in said B-mode image.

3. The ultrasonic image display system as recited in claim 1, wherein:
said at least one B-mode image is first and second B-mode images displayed side by side,
in said first B-mode image is displayed said first measured portion, and
in said second B-mode image is displayed said second measured portion.

4. The ultrasonic image display system as recited in claim 1, wherein: said first and second measurements include any of measurements relating to elasticity, viscosity, a subcutaneous thickness, and ultrasound attenuation in biological tissue in said patient, a measurement of asperities of a patient's liver surface, and analysis on texture in said B-mode image(s).

5. The ultrasonic image display system as recited in claim 4, wherein: said first measurement is a measurement relating to elasticity of biological tissue in said patient, and said second measurement is a measurement of ultrasound attenuation in the biological tissue in said patient.

6. The ultrasonic image display system as recited in claim 5, wherein: the measurement relating to elasticity of biological tissue in said patient is a measurement for acquiring a velocity of propagation of shear waves generated in said biological tissue by a push pulse transmitted to said biological tissue.

7. The ultrasonic image display system as recited in claim 6, wherein: said processor further displays in said first measured portion an elasticity image having colors depending upon said velocity of propagation.

8. The ultrasonic image display system as recited in claim 5, wherein: the measurement relating to elasticity of biological tissue in said patient is a measurement for acquiring a velocity of propagation of shear waves generated in said biological tissue by a push pulse transmitted to said biological tissue to acquire an elasticity value of said biological tissue based on said velocity of propagation.

9. The ultrasonic image display system as recited in claim 8, wherein: said processor further displays in said first measured portion an elasticity image having colors depending upon said elasticity value.

10. The ultrasonic image display system as recited in claim 1, wherein: before said first measuring scan, said processor controls said ultrasonic probe to perform an additional B-mode scan on said patient, and refreshes the B-mode image in which said first measured portion is displayed.

11. The ultrasonic image display system as recited in claim 1, wherein: before said second measuring scan, said processor controls said ultrasonic probe to perform an additional B-mode scan on said patient, and refreshes the B-mode image in which said region is displayed.

12. The ultrasonic image display system as recited in claim 1, wherein: said first measuring scan is performed so as to include at least a portion of said patient that corresponds to said first measured portion, and said second measuring scan is performed so as to include at least a portion of said patient that corresponds to said region in said patient.

13. A program for controlling an ultrasonic image display system comprising a processor, an ultrasonic probe, a display, memory, and a user interface, wherein
said program for controlling is adapted to cause said processor to perform control comprising the acts of:
controlling said ultrasonic probe to perform a B-mode scan for a B-mode image on a patient;
displaying on said display at least one B-mode image based on echo data obtained by said B-mode scan;
displaying in said B-mode image a first measured portion serving as an object of a first measurement and a region indicating a candidate of a second measured portion serving as an object of a second measurement;
after said first measured portion has been displayed, controlling said ultrasonic probe to perform a first measuring scan for said first measurement on said patient, and acquiring a result of the first measurement for said first measured portion based on echo data obtained by said first measuring scan; and
after said region has been displayed, controlling said ultrasonic probe to perform a second measuring scan for said second measurement on said patient, and storing echo data obtained by said second measuring scan in said memory,
said user interface is adapted to, after the echo data obtained by said second measuring scan has been stored in said memory, accept an operator's input for defining said second measured portion in said region, and
said program for controlling is further adapted to cause said processor to perform the act of, once said user interface has accepted said operator's input, acquiring a result of the second measurement for said second measured portion based on the echo data that has been obtained by said second measuring scan and stored in said memory.
